(19) United States
(12) Patent Application Publication
(10) Pub. No.: US 2009/0075383 A1
(43) Pub. Date: Mar. 19, 2009

(54) COMPOSITION AND METHOD FOR EFFICIENT DELIVERY OF NUCLEIC ACIDS TO CELLS USING CHITOSAN

(75) Inventors: Michael D. Buschmann, Montreal (CA); Marc Lavertu, Pointe-Claire (CA); Stephane Methot, Montreal (CA)

Correspondence Address: DAVID S. RESNICK, NIXON PEABODY LLP, 100 SUMMER STREET, BOSTON, MA, 02110-2131, US

(73) Assignee: BIO SYNTECH CANADA INC., Laval, QC (CA)

(21) Appl. No.: 12/092,498

(22) PCT Filed: Nov. 6, 2006
(86) PCT No.: PCT/CA06/01813

§ 371 Date: Oct. 20, 2008

Related U.S. Application Data
(60) Provisional application No. 60/733,173, filed on Nov. 4, 2005.

(51) Int. Cl. C12N 15/85

(52) U.S. Cl. 435/455

(57) ABSTRACT

There is disclosed a composition and a method for the efficient non-viral delivery of nucleic acids to cells using chitosan. In order to achieve high efficiency of transfection, the composition contains a nucleic acid and a chitosan that has the following physico-chemical properties: a combination of a number-average molecular weight between 8 kDa and 185 kDa and a degree of deacetylation between 72% and 92%. The chitosan molecule can also present additional physiochemical properties such as a block distribution of acetyl groups obtained by a heterogeneous treatment of chitin, and/or a polydispersity index between 1.4 and 7.0. By correctly controlling these parameters, efficient delivery systems may be produced that are effective when optimized for different conditions such as the pH of transfection media and amine-to-phosphate ratio.

### CROSS-REFERENCE TO RELATED APPLICATIONS

This application claims priority on U.S. application Ser. No. 60/733,173 filed Nov. 4, 2005, the entire content of which is hereby incorporated by reference.

### FIELD OF THE INVENTION

The invention relates to an improved (optimized) composition and method for the efficient non-viral delivery of nucleic acids to cells using chitosan.

### BACKGROUND OF THE INVENTION

### 1) The Nucleic Acid Delivery Problem

### Viral and Non-Viral Vectors

Gene therapy consists of the introduction and expression of genetic information in cells to achieve a particular therapeutic effect such as curing a disease or slowing its progression or regenerating damaged tissues. A delivery vehicle, referred to as a vector, of viral or non-viral origin, is required to condense and carry the therapeutic DNA into the target cells. Viral systems present high delivery and expression efficiencies as they are natural highly evolved DNA carriers. However, safety issues for viral vectors have limited their clinical use. Viral vectors can produce endogenous recombination, oncogenic effects and immunological reactions leading to potentially serious complications. Moreover, viral vectors have limited DNA carrying capacity, production and packaging problems and are expensive to produce. Non-viral vectors possess the important advantage of being non-pathogenic and non-immunogenic. These vectors are also easier and less expensive to produce and have a larger DNA carrying capacity. However, their delivery and expression efficiencies are relatively low compared to viral systems. There are two main challenges to overcome in order to establish an effective non-viral-based gene therapy system: 1) The development of DNA constructs that provide long-term expression of therapeutic genes and 2) The development of suitable and efficient methods to deliver vector DNA to target cells. The current invention addresses this latter requirement.

### 2) Non-Viral Vectors for Nucleic Acid Delivery

The chemical methods of non-viral gene delivery include calcium phosphate precipitation, cationic lipids and cationic polymers (MacLaughlin, F. C. et al., J. Control. Release 56: 259-272, 1998). Naked DNA can also be delivered where its main route of administration being intramuscularly. Cationic compounds are the most promising among the non-viral vectors as they have shown relatively high efficiency.

### Naked DNA

In 1990, it was reported that muscle cells can be transfected and express genes after intramuscular injection of plasmid DNA, as disclosed in U.S. Pat. No. 5,580,859. Mumper and Rolland developed what they termed a protective interactive, non-condensing (PINC) delivery system designed to complex plasmid DNA to facilitate the uptake of naked plasmid by muscle as compared to plasmid formulated in saline, as disclosed in U.S. Pat. No. 6,514,947. Some of these PINC systems formulations showed up to a 10 fold increase of the level of expression over the plasmid formulated in saline.

### Calcium Phosphate

Calcium phosphate precipitates have a limited efficiency and cannot be used in vivo since they do not protect DNA from DNAse degradation. However, it is now possible to protect plasmid DNA from an external DNAse environment by encapsulating the DNA inside the calcium phosphate nanoparticles. These nanoparticles presented a modest increase in transfection efficiency in vitro in comparison to the standard calcium-phosphate precipitation technique. The calcium-phosphate complexes are known to be relatively non-toxic.

### Cationic Lipids

Cationic lipid-nucleic acid complexes (lipoplexes) are formed by the electrostatic interaction of anionic nucleic acids binding to the surface of cationic liposomes eventually forming multilamellar lipid-nucleic acid complexes. Since the first cationic lipid DOTMA (N-[1-(2,3,-dioleyloxy)propyl]-N,N,N-trimethylammonium chloride), many cationic lipids have been developed. Lipoplexes are one of the most efficient ways of delivering nucleic acids into cultured cells and are increasingly being used in vivo. There are currently more than 30 different commercial varieties of cationic formulations available. The liposome formulations usually include a cationic lipid and a neutral lipid such as DOPE (dioleoylphosphatidylethanolamine) that is commonly used. The neutral lipid contributes to the stabilization of the cationic liposome formulation and facilitates membrane fusion as well as contributing to the destabilization of the plasmalemma or endosome. Varying the ratio of cationic to neutral lipid of the liposome formulation can change the level of transfection.

A serious drawback of lipoplexes is their toxicity, as observed in cultured cells and confirmed as well by several in vivo findings. In addition, these complexes exhibit an immunostimulation effect that may either be harmful or beneficial. The toxicity of lipoplexes is reported to be closely associated with the charge ratio of cationic lipid to nucleic acid in the formulation. The type of formulation used and the dose of lipoplexes administered also influence toxicity. Higher charge ratios of cationic lipid to nucleic acid are generally more toxic to a variety of cell types, including cancer cell lines. Due to this toxicity, the in vivo delivery of lipoplexes must be as close in proximity to the target site as possible to minimize side effects. More biocompatible formulations are being tested in order to reduce the toxicity of lipoplexes. For example, the in vitro toxicity of lipid based formulation have been reduced by grafting synthesized cationic poly(ethylene glycol) (PEG) lipids on nearly neutral “stabilized plasmid-lipid particles” (SPLP). The level of transfection achieved with this formulation in baby hamster kidney (BHK) cells was found to be significantly improved with increasing concentration of Ca²⁺.

### Cationic Polymers

The principle behind the use of polycations for DNA delivery is that the oppositely charged polycation and DNA interact strongly to form precipitated particles (polyplexes) of nanometric size to encapsulate the DNA and protect it from nuclease activity that can degrade DNA in seconds. Most often an excess of polycation is used (Romoren, K. et al., Int. J. Pharm. 261: 115-127, 2003), such that the particle bears a net positive charge to aid its non-specific binding to the plasma membrane. Many polyplexes using cationic polymers have superior transfection efficiency and lower serum sensitivity compared to lipoplexes. A large number of natural and synthetic cationic polymers have been used as vehicle for gene delivery. Among naturally occurring polycations are proteins such as histones, cationized human serum albumin, as well as aminopolysaccharides such as chitosan. The group of synthetic polycations includes peptides such as poly-L-lysine (PLL), poly-L-ornithine, and poly(4-hydroxy-L-proline ester), as well as polyamines such as polyethylenimine (PEI), polypropylenimine, and polyamidoamine dendrimers. Linear and dendritic poly(b-aminoesters) have been synthesized and appear to be efficient gene delivery vectors. There are also all the various derivatives of some of the vectors listed above that are being developed to improve efficiency and specificity as well as to reduce toxicity. The most studied cationic polymer-based delivery systems are PEI, PLL, chitosan, and polyamidoamine, ranked with respect to the number of reported studies, making chitosan the most studied natural polycation.

An advantage of polyplexes is that their formation does not require interaction of multiple polycations, contrary to the need for multiple lipid components in liposomes, so that their macroscopic properties are easier to control. Adjuvants are also generally not required for polyplexe preparation. Another advantage of polycations is that being formed of repeating structural units, they can be directly chemically modified to obtain higher efficiency or cell targeting. However, despite these advantages, many cationic polymers have been found to be toxic, possibly arising from interactions with plasma membrane. Several cationic polymers were ranked according to their toxicity as follows: PEI=PLL>poly(diallyl-dimethyl-ammonium chloride) (DADMAC)>diethylaminoethyl-dextran (DEAE-dextran)>poly(vinyl pyridinium bromide) (PVPBr)>PAMAM N cationic human serum albumin (cHSA)>native human serum albumin (nHSA). Moreover, PEI, DADMAC and PLL tested with red blood cells were found to be highly damaging to plasma membranes. There are many possible sources of cytotoxicity. Surface charge density may be involved since high charge density polyplexes show higher toxicity. Furthermore, it has been reported that the charge density in the polymer plays a more important role in cytotoxicity than the total amount of charge. Toxicity may be molecular weight dependent as well, since the cytotoxicity of PEI increases linearly with molecular weight. Accumulation of non-degradable polymers such as PEI in the lysosome (“Lysosomal loading”) may yet be an additional contributor to toxicity.

### 3) Chitosan as a Vector for Nucleic Acid Delivery

Chitin, found mainly in crustacean shells, is thought to be the second most abundant natural polysaccharide after cellulose. It is a linear homopolymer composed of β-1,4-linked N-acetyl-glucosamine, from which chitosan is derived by a process of alkaline deacetylation resulting in a polysaccharide composed of glucosamine and N-acetyl-glucosamine monomers linked by β-1,4 glycosidic bonds. The molecular weight (MW) of chitosan as well as the amount of amine groups (degree of deacetylation or DDA) on the chain have an influence on its biological and physicochemical properties (Huang, M. et al., Pharm. Res. 21: 344-353, 2004; Zhang, H. and Neau, S. H., Biomaterials 22: 1653-1658, 2001). For example, the amount and distribution of acetyl groups affects biodegradability since the absence of acetyl groups or their homogeneous distribution (random rather than block) results in very low rates of enzymatic degradation.

Chitosan has attracted attention in the pharmaceutical and biomedical fields since it possesses well known beneficial biological properties including biocompatibility (Richardson, S. C. et al., Int. J. Pharm. 178: 231-243, 1999), low toxicity, biodegradability, mucoadhesiveness, haemostatic ability, and antimicrobial/antifungal activities. Moreover, chitosan is a polycation and is thus able to package DNA in solution by a process of coacervation, making it a useful non-viral gene delivery vector. Chitosan is one of the most widely used non-viral vectors in the family of cationic polymers for DNA packaging and condensation (Ishii, T. et al., Biochim. Biophys. Acta 1514: 51-64, 2001; Kiang, T. et al., Biomaterials 25: 5293-5301, 2004; Koping-Hoggard, M. et al., J. Gene Med. 5: 130-141, 2003; Koping-Hoggard, M. et al., Gene Ther. 8: 1108-1121, 2001; Koping-Hoggard, M. et al., Gene Ther. 11: 1441-1452, 2004; Leong, K. W. et al., J. Control. Release 53: 183-193, 1998; MacLaughlin, 1998, supra; Mao, H. Q. et al., J. Control. Release 70: 399-421, 2001; Richardson, 1999, supra; Romoren, 2003, supra; Sato, T. et al., Biomaterials 22: 2075-2080, 2001), along with polylysine and polyethyleneimine. Cellular internalization of chitosan appears to occur via fluid phase macropinocytosis.

The use of chitosan as a non-viral gene transfer vector is becoming more popular as the knowledge acquired from in vitro studies on different cell lines is being translated to in vivo animal models. For example, DNA-based immunization has been reported through oral, nasal and topical routes using chitosan-based vectors. Fortunately, chitosan does not possess the cytotoxic effects of many other non-viral systems but has demonstrated rather low toxicity, both in vitro and in vivo (Koping-Hoggard, 2001, supra).

In addition to possible toxicity, it is important to account for carrier-alone induced changes in gene expression, as for polyethylenimine and cationic lipids. For example, pronounced cellular effects are seen when monocytes are exposed to chitosan, however contradictory data exist in the literature, where some studies report direct stimulation of TNF-α release others none, and yet others that chitosan prevents LPS from inducing TNF-α release.

Earlier studies of gene transfer with chitosan often report the molecular weight of the chitosan used in approximate terms: oligomers (Koping-Hoggard, 2003, supra; Koping-Hoggard, 2004, supra), low (Sato, 2001, supra), intermediate (Huang, 2004, supra; MacLaughlin, 1998, supra; Sato, 2001, supra) and high molecular weight (MacLaughlin, 1998, supra; Mao, 2001, supra). Kiang et al. (Kiang, 2004, supra) even used chitosans of different degrees of deacetylation produced by heterogeneous acetylation of three starting chitosans with molecular weights 138 kDa, 209 kDa and 390 kDa. The influence of the degree of deacetylation on gene expression was however only examined using the 390 kDa-chitosan at DDA levels of 90%, 70% and 62%, and, since the molecular weight is very high, could not observe any interaction between molecular weight and degree of deacetylation of chitosan in determining transfection efficiency. It was found that lowering the DDA to 70% and 62% decreased luciferase transgene expression in HEK 293, SW 756 and HeLa cells. This result was attributed to either a decreased stability of the nanoparticles at the lower DDAs or an increased susceptibility to enzymatic degradation due to a less compact state.

Only recently have some studies attempted to examine both the molecular weight and the degree of deacetylation together as important contributors determining the level of gene expression. Romoren et al. (Romoren, 2003, supra) studied the effect of selected formulation variables, including molecular weight and degree of deacetylation, on the in vitro transfection efficiency on EPC cells using an experimental design in combination with multivariate data analysis. Results suggested that the charge ratio of amine (chitosan) to phosphate (DNA) is the most strongly positively correlated parameter followed by molecular weight and DNA concentration, while the degree of acetylation (F_{A}; the inverse of DDA) was negatively correlated. Interactions between the MW and charge ratio, as well as between F_{A} and the charge ratio were suggested but no relationship was found between MW and DDA (or F_{A} in this study). Two favorable formulations were identified solely based on molecular weight and charge ratio, however no standard reference such as a commercial phospholipids system was used to evaluate transfection efficiency relative to such a standard.

Huang et al. (Huang, 2004, supra) studied the effects of MW and DDA of chitosan on nanoparticles uptake and cytotoxicity on A549 cells, but did not examine transfection efficiency in this study since the nanoparticles were prepared by ionotrophic gelation of chitosan with pentasodium tripolyphosphate (TPP) without the addition of DNA. Chitosan alone or condensed with the polyanionic TPP showed the same cytotoxicity profile which was attenuated by decreasing DDA, and to a lesser extent, by diminishing the MW. Nanoparticle uptake was a saturable event for all chitosan samples studied (chitosans of 10, 17, 48, 98, and 213 kDa at 88% DDA and 213 kDa at 46% and 61% DDA). A decreasing MW and DDA diminished the nanoparticle binding affinity and uptake capacity, and was correlated with the zeta potential of the complexes.

Recently, Huang et al. (Huang, M., et al., Journal of Controlled Release 106:391-406, 2005) studied the effect of chitosan molecular weight and degree of deacetylation on uptake, nanoparticle trafficking and transfection efficiency on A549 cells. However, his study only used 7 formulations (chitosan of 10, 17, 48, 98 and 213 kDa at 88% DDA; 213 kDa at 61 and 46% DDA), most of which were at a single value of DDA (88%), to study the effect of MW and DDA on transfection efficiency. They found that a decrease in MW and DDA apparently renders lower transfection efficiency. However, as described in the current invention, the relationship between those two parameters is much more complex and demands an equilibrium between those two parameters to achieve optimal stability that translates into a most favorable transfection efficiency. Due to their limited number of formulations, they could not observe this complex relationship between MW and DDA. Moreover, only one parameter at a time was varied preventing them to see a coupling effect between MW and DDA in relation with the pH of the transfection media and the chitosan-to-DNA ratio (commonly referred to the amine-to-phosphate or N:P ratio), as described in the current invention.

Chitosan was used to deliver a pharmacologically active compound such as insulin through an intranasal route in the rat and sheep with a formulation in the form of a solution (WO 90/09780, U.S. Pat. No. 5,554,388, U.S. Pat. No. 5,744,166). The chitosan/insulin formulations were prepared by mixing equal volumes of insulin and chitosan in solution. These formulations involved the use of a water-soluble chitosan of molecular weights of 10 kDa or greater, preferably at least 100 kDa or 200 kDa and most preferably about 500 kDa, with no specification on degree of deacetylation.

Chitosan was also used as an adjuvant for the immunization of mice through an intranasal route with a soluble formulation (US publication 2003/0039665). These formulations involved a water-soluble chitosan glutamate of molecular weights between 10-500 kDa, preferably between 50-300 kDa and more preferably between 100-300 kDa with a degree of deacetylation greater than 40%, preferably between 50-90%, and more preferably between 70-95. Chitosan, normally soluble in a weak acid can be modified in different ways, such as chitosan glutamate, to make it water-soluble.

The in vitro transfection of rabbit synoviocytes and the in vivo expression in the intestinal mucosa of rabbits after oral administration was assessed using formulations involving chitosans and chitosan oligomers (8, 13, 22, 41, 70 and 90 kDa) and an endosomolytic peptide. The formulations used for those studies comprise a chitosan-based compound with a molecular range of 5-1000 kDa and a nucleic acid or oligonucleotide where a cryoprotectant is added, as disclosed in WO 97/42975, U.S. Pat. No. 6,184,037 and US 2001/0031497. In those patent applications or patents, no appreciation of the degree of deacetylation was reported except for a small variation from 69-79% that was related to the nitrous acid deamination of the same starting material. In addition, reasons for this variability of degree of deacetylation was not understood.

Fully de-N-acetylated chitosan (DDA 100%) of low molecular weight (oligomers<10 kDa) was branched with either oligosaccharides, D-glucose or acetaldehyde and subsequently used (branched or not) in formulations with DNA for cellular transfection of an epithelial human embryonic kidney cell line (HEK 293). The chitosan involved in the formulations was claimed to have a fraction of N-acetyl-D-glucosamine residues (F_{A}) between 0-0.7, preferably between 0-0.35, more preferably between 0-0.1 and most preferably between 0-0.01, corresponding to the following degree of deacetylation (DDA): 30-100%, 65-100%, 90-100% and 99-100%. Its degree of polymerization was 2-2500, preferably 3-250, and most preferably 4-50, corresponding approximately to the following molecular weights: 320-400,000 Da, 480-40,000 Da and 640-8,000 Da. Moreover, 1-60% of D-glucosamine residues of the said chitosan carried branching groups, preferably 2-40%, and most preferably 3-20%, as disclosed in WO 03/092740.

Monodisperse fully de-N-acetylated chitosan oligomers, obtained by fractionation using size-exclusion chromatography, were used in formulations with DNA for in vitro gene transfer to the HEK 293 cell line and for in vivo gene expression in the lung of mice when administered in the form of a solution in the surgically-opened trachea. The fully N-deacetylated chitosan oligomers involved in the formulations contained a weight fraction of less than 20% of oligomers with a degree of polymerization (DP)<10 in addition to a weight fraction of less than 20% with DP>50, preferably less than 20% DP<12 and less than 20% DP>40, most preferably less than 20% DP<15 and less than 20% DP>30.

Solid nanospheres of chitosan/DNA of sizes between 200-300 nm, and less than 151 nm were formed by coacervation in the presence of sodium sulfate (5-100 mM) with an optional linking moiety or a targeting ligand attached to the surface, and were used for cell transfection. The physicochemical properties of the chitosan involved in this formulation were not specified, as disclosed in WO 98/01162 and U.S. Pat. No. 5,972,707. Moreover, these details about shape and size of the chitosan/DNA formulations are not essential since the data in the present invention shows that efficient transgene expression occurs with different particle sizes with no apparent preference for small chitosan/DNA particles.

As indicated in the literature, many of the biological effects of chitosan are strongly dependent on molecular weight and acetyl content and distribution, parameters that have often been incompletely defined in many of previous studies. It is clear that chitosans must be carefully prepared and precisely characterized in order to relate biological responses to molecular parameters.

As can be seen from the above, chitosan has been used as non-viral transfer or delivery vector. However many conditions may affect its utility and efficiency, and these conditions were still poorly understood to date until the present invention that they must be considered together in case one condition affects another one, such that the commercial use of chitosan to date is inexistent due to its inefficiency (from a commercial point of view) to deliver nucleic acid sequences.

### SUMMARY OF THE INVENTION

Many have tried and used so far without much success chitosan as a non-viral vector. However, no one ever considered optimizing the chitosan composition as it is now reported herein. More particularly, no one ever considered that by varying the molecular weight, without adjusting accordingly the degree of deacetylation and optionally the amine:phosphate ratio and/or the pH, the efficacy of the transfer vector may be affected. With such optimization as reported herein, commercial use may now be considered a viable option to other transfer vectors such as lipofectamine, which is one efficient vector broadly used although its toxicity is the main limiting factor unlike chitosan.

The present invention relates to a composition and method for the efficient non-viral delivery of nucleic acids to cells using the natural polysaccharide chitosan. The composition contains a nucleic acid and a chitosan that has the following physicochemical properties: the combination of a number-average molecular weight (Mₙ) between 8 kDa and 185 kDa and a degree of deacetylation between 72% and 92%. The chitosan molecule can also present additional physicochemical properties such as a block distribution of acetyl groups obtained by a heterogeneous treatment of chitin, and/or a polydispersity index (PDI=M_{w}/Mₙ, where M_{w} is the weight average molecular weight) between 1.4 and 7.0. A method for delivering a nucleic acid to cells by applying this composition is also provided.

It has been found in the present invention that the range of intermediate number average molecular weight chitosan from 8 kDa to 185 kDa can be effective gene transfer vectors when combined with plasmid DNA provided that an appropriate degree of deacetylation between 72% and 92% of the chitosan is chosen as a function of molecular weight, medium pH and the ratio of chitosan to plasmid DNA. The invention therefore provides compositions and methods to achieve high levels of gene transfer and subsequently, gene expression, which was not previously recognized in prior art using chitosan and a nucleic acid. According to the present invention, chitosan of intermediate molecular weight can be efficient gene transfer vehicles if DDA is appropriately controlled. By correctly accounting for these two parameters molecular weight and DDA in a combined fashion, efficient delivery systems may be produced that are effective when optimized for different conditions (pH of transfection media and amine-to-phosphate ratio).

In accordance with the present invention, there is thus provided a composition comprising chitosan and a nucleic acid sequence for delivery of said nucleic acid sequence into cells, wherein the chitosan has a number-average molecular weight (Mₙ) between 8 kDa and 185 kDa and a degree of deacetylation between 72% and 92%.

In accordance with the present invention, there is also provided a method for delivering a nucleic acid sequence into a cell comprising the step of contacting the composition of the present invention with said cell.

Still in accordance with the present invention, there is also provided the use of the composition of the present invention for delivering nucleic acid sequences or fragments to a cell.

### BRIEF DESCRIPTION OF THE DRAWINGS

FIG. 1: Percentage of HEK 293 cells that were transfected in vitro using complexes made with chitosans BST-80 (40 kDa) and BST-72 (80 kDa). Cells were incubated 48 hours in 24-well culture plates with complexes at A) Different amine:phosphate (N:P) ratios with fixed 2.5 μg of pDNA/well and fixed transfection media pH of 7.1; B) Different pH with fixed N:P ratio of 7:1 and fixed 2.5 μg of pDNA/well; C) Different amounts of pDNA/well with fixed pH of 7.1 and fixed N:P of 7:1.

FIG. 2: Size of chitosan/pDNA complexes made with chitosans of different degrees of deacetylation and molecular weights. Two different N:P ratios were used (5:1 and 10:1) as well as two different pH (6.5 and 7.1) of the suspension buffer (PBS) in which size was measured.

FIG. 3: Zeta potential of chitosan/pDNA complexes made with chitosans of different degrees of deacetylation and molecular weights. Two different N:P ratios were used (5:1 and 10:1) as well as two different pH (6.5 and 7.1) of the suspension buffer (PBS) in which zeta potential was measured.

FIG. 4: Percentage of HEK 293 cells that were transfected in vitro using complexes made with chitosans of different degrees of deacetylation and molecular weights. Cells were incubated for 48 hours in 24-well culture plates with complexes made with amine:phosphate ratios of 5:1 and 10:1, using transfection media pH of 6.5 and 7.1 as well as fixed 2.5 μg of pDNA/well. FuGENE 6, the positive control resulted in 77.7±2.6% at pH 6.5 and 82.6±2.3% at pH 7.1.

FIG. 5: Transgene expression by HEK 293 cells transfected in vitro using complexes made with chitosans of different degrees of deacetylation and molecular weights. Cells were incubated for 48 hours in 24-well culture plates with complexes made with amine:phosphate ratios of 5:1 and 10:1, using transfection medium pH of 6.5 and pH 7.1, and fixed 2.5 μg of pDNA/well. The relative light units (RLU) were normalized to the protein content of each sample. The different formulations were compared to control cells (C), pDNA alone (D) as a negative control as well as Lipofectamine™ (L) and FuGENE 6 (F) as positive controls. An asterisk (*) indicates similar expression levels since a Mann-Whitney test with p=0.05 showed no significant difference.

FIG. 6: Transgene expression quantified by a luciferase assay correlated with the percentage of transfected cells measured by flow cytometry detection of GFP using a plasmid containing both reporters. The pH of the transfection medium needs to be accounted for when relating these two parameters. Linear Regression resulted in Pearson product moment correlation coefficients of 0.91, p<0.0001 (pH 6.5) and 0.94, p<0.0001 (pH 7.1). Dashed lines display 95% confidence intervals.

FIG. 7: Contour plot of normalized transgene expression of HEK 293 cells as a function of degree of deacetylation (DDA) and molecular weight (MW). In each plot, transgene expression was normalized to the highest expression level obtained with this particular N:P ratio and transfection medium pH.

FIG. 8: Schematic summary of complex stability, and resulting transfection efficiency, as a function of molecular weight (MW) and degree of deacetylation (DDA) of chitosan. An optimal stability window exists that will yield efficient transfection for a DDA versus MW region that depends on particular values of pH and N:P ratio.

FIG. 9: Percentage of transfected cells using different formulations of chitosan/DNA nanoparticle in three cell lines, namely Caco-2, HeLa and HT29, is reported herein.

FIG. 10: In vivo FGF-2 protein expression in balb/c mice after sub-cutaneous vaccination is reported using different formulations of chitosan/pVax-4sFGF-2.

FIG. 11: In vivo FGF-2 antibody production in balb/c mice after sub-cutaneous vaccination is reported using different formulations of chitosan/pVax-4sFGF-2.

### DETAILED DESCRIPTION OF THE INVENTION

Deuterium oxide (Cat #15, 188-2), deuterium chloride 20% (w/v) in deuterium oxide (Cat #17, 672-9), sodium nitrite (Cat #431605), hydrochloric acid (Cat #31, 894-9) and glacial acetic acid (Cat #33, 882-6) were purchased from Aldrich. Sodium azide (Cat #S2002), HEPES (Cat #H-4034), MES (Cat #M-2933) and 1N sterile filtered HCl (Cat #H9892) were purchased from Sigma. Anhydrous sodium acetate, Omnipur (Cat #EM7510) was purchased from VWR. Sodium hydroxide (Cat #S320-1) was purchased from Fisher. HEK 293 cells were purchased from ATCC (ATCC #CRL 1573). DMEM high glucose (DMEM HG, Cat #12100-046), Fetal Bovine Serum (FBS, Cat #26140-079), Lipofectamine™ (Cat #18324-111), Trypsin-EDTA (Cat #2500-056) and Competent DH5α cells (Cat #182630-12) were purchased from LifeTechnologies. FuGENE 6 Transfection Reagent (Cat #1815091) was purchased from Roche Diagnostics. Bright-Glo™ Luciferase Assay System (Cat #E2620) and Glo Lysis Buffer (Cat #E2661) were purchased from Promega. BCA™ Protein Assay Kit (Cat #23227) and Compat-Able™ Preparation Reagent Set (Cat #23215) were purchased from Pierce Biotechnology. The plasmid EGFPLuc was purchased from Clontech (Cat #6169-1). The EndoFree Plasmid Mega Kit (Cat #12381) was purchased from Qiagen.

Ultrapure chitosan samples (Ultrasan™) were provided by Bio Syntech Inc. (Laval Qc., Canada) where quality controlled manufacturing processes eliminate contaminants including proteins, bacterial endotoxins, toxic metals, inorganics and other impurities. All chitosans had less than 500 EU/g of bacterial endotoxins. Chitosans were selected to have a range of degree of deacetylation from 98-72% and these bulk batches were named accordingly (Table 1). These chitosans were produced by heterogeneous deacetylation resulting in a block rather than random distribution of acetyl groups.

TABLE 1
Physicochemical Characteristics of Bulk Chitosans

| | Degree of | Number-average | |
|---|---|---|---|
| | deacetylation | molecular weight | Polydispersity |
| Chitosans | DDA (%)^{a} | Mₙ (kDa)^{b} | PDI (M_{w}/Mₙ) |
| BST-98 | 98 | 120 | 1.5 |
| BST-92 | 92 | 200 | 1.4 |
| BST-80 | 80 | 320 | 1.5 |
| BST-72 | 72 | 220 | 1.5 |

^{a}Determined by ¹H NMR.
^{b}Determined by gel permeation chromatography.

Chitosans of different DDA were depolymerized using nitrous acid to achieve specific number-average molecular weight targets (Mₙ) of 10, 40, 80 and 150 kDa, except for BST-98 which already had a starting Mₙ of 120 kDa, that therefore replaced the 150 kDa chitosan for 98% DDA. For depolymerization, chitosans were dissolved overnight at 0.5% (w/v) in 50 mM hydrochloric acid under magnetic stirring and then treated for 16 hours at room temperature with specific amounts of sodium nitrite in the range of 0.001-0.1 mole per mole of chitosan glucosamine. The reaction was stopped by precipitation using 6N sodium hydroxide to bring the pH above 10. Chitosans were then washed by repeated centrifugation (4000 g for 2 min) and resuspended in deionized water, until the supernatant reached neutral pH. The samples were freeze-dried prior to characterization and use in the production of nanoparticles.

Number- and weight-average molecular weights (Mₙ and M_{w}) of chitosans were determined by gel permeation chromatography (GPC) using a Hewlett Packard Series 1100 chromatographic system equipped with a refractive index detector (Agilent technologies Inc., Mississauga ON, Canada) combined with a Viscotek T60A dual detector (Viscotek, Houston Tex., USA) containing light scattering and viscometer detectors. Dry chitosan powder was dissolved in duplicate for 24 hours on a rotary mixer (Labquake®, Barnstead International Inc., Dubuque Iowa, USA) at 1 mg/ml in the mobile phase consisting of acetic acid 0.3 M, sodium acetate 0.2 M and sodium azide 0.8 mM, pH=4.5. The solutions were filtered prior to injection using a 0.45 μm nylon membrane syringe filter (Life Science, Petersborough ON, Canada). The samples were then run on an analytical SEC polymer-based linear (mixed-bed) column (TSK-Gel GMPWXL, Viscotek) at a flow-rate of 0.8 mL/min and a column temperature of 25° C. The system was calibrated with a narrow standard, PolyCAL Polyethylene Oxide-PEO26K (Viscotek) and subsequently validated with a broad standard PEOX500K (Mₙ 180.6 kDa, M_{w} 475.5 kDa; American Polymer Standards, Mentor Ohio, USA). Integration boundaries were set using TriSEC GPC software (Viscotek) by manual inspection of the elution profile and were always set by the same analyst.

Degree of deacetylation was determined by ¹H NMR according to Lavertu et al. (Lavertu, M. et al., J. Pharm. Biomed. Anal. 32: 1149-1158, 2003). Briefly, chitosan solutions were prepared by stirring, at room temperature, 10 mg of chitosan in 1.96 ml of D₂O containing 0.04 ml of DCl for 30 minutes to ensure complete dissolution of the polymer. After dissolution, approximately 1 ml of the chitosan solution was transferred to a 5 mm NMR tube. The sample tube was inserted in the magnet and allowed to reach thermal equilibrium at 70° C. (10 minutes) before performing the experiment. ¹H NMR spectra were acquired on a Varian Mercury 400 MHz spectrometer as described previously (Lavertu, 2003, supra). As measured by Lavertu et al., this technique gives an excellent precision on DDA measurements with a coefficient of variation less than 0.8%.

The plasmid EGFPLuc of 6.4 kb (Clontech Laboratories) encodes for a fusion of enhanced green fluorescent protein (EGFP) and luciferase from the firefly Photinus pyralis, driven by a Human cytomegalovirus (CMV) promoter. This plasmid was amplified in DH5α bacteria and purified using the EndoFree Plasmid Mega Kit (Qiagen). The purified pDNA was dissolved in endotoxin-free tris-EDTA (TE) and concentration/purity determined by UV spectrophotometry by measuring absorbance at 260/280 nm.

Depolymerized chitosans were dissolved overnight on a rotary mixer at 0.5% (w/v) in hydrochloric acid using a glucosamine:HCl ratio of 1:1. Chitosan solutions were then diluted with deionized water to reach the desired amine (deacetylated groups) to phosphate ratio when 100 μL of chitosan would be mixed with 100 μL of pDNA, the latter always at a concentration of 330 μg/mL in endotoxin-free tris-EDTA (TE). Prior to mixing with pDNA, the diluted chitosan solutions were sterile filtered with a 0.2 μm syringe filter and ninhydrin assays indicated that chitosan was not trapped in the filter. Chitosan/pDNA nanoparticles were then prepared by adding 100 μL of the sterile diluted chitosan solution to 100 μL of pDNA (330 μg/mL) at room temperature, pipetting up and down and tapping the tubes gently. Chitosan/pDNA nanoparticles were then used for transfection 30 minutes after preparation.

HEK 293 cells were cultured in DMEM HG with 1.85 g/L of sodium bicarbonate and supplemented with 10% FBS at 37° C. and at 5% CO₂. Cells were subcultured according to ATCC recommendations without any antibiotics. The absence of mycoplasma was verified by fluorescence detection. For transfection, HEK 293 cells were plated in 24-well culture plates using 500 μl/well of complete medium and 50,000 cells/well, incubated at 37° C., 5% CO₂. The cells were transfected the next day at ˜50% confluency.

Complete transfection media were equilibrated overnight at 37° C. and 5% CO₂ and pH adjustment was performed with 1N sterile HCl just before transfection. In order to increase pH stability of transfection media, HEPES (for pH 7.1 and 7.4) and MES (for pH 6.5 and 6.8) were added to DMEM HG and sodium bicarbonate concentration was decreased accordingly. Chitosan/pDNA complexes were prepared, as described above, 30 minutes before being incubated with cells. Medium over cells was then aspirated and replenished with 500 μl transfection medium containing chitosan/pDNA complexes at a concentration of 2.5 μg pDNA/well, unless otherwise noted. Cells were incubated with chitosan/pDNA complexes until analysis at 48 hours post-transfection. Cells were then observed under a fluorescence microscope (Zeiss Axiovert) to monitor any morphological changes and to obtain an estimate of the transfection efficiency. Transfection efficiencies and transgene expression levels were quantitatively assessed by flow cytometry and luciferase assay, respectively. FuGENE 6 and Lipofectamine™ were used as positive controls and uncomplexed naked pDNA was used as a negative control. All experiments were done in duplicates, with a minimum of three separate experiments to demonstrate reproducibility.

FuGENE 6/pDNA complexes were prepared with a 1:3 ratio of pDNA(μg):FuGENE 6(μl), according to manufacturer specifications and were used as a positive control. The transfection medium was identical to that of chitosan. Cells were incubated for 48 hours with FuGENE 6/pDNA complexes (2.5 μg pDNA/well) until analysis.

Lipofectamine™/pDNA complexes were prepared with a 1:2 ratio of pDNA(μg):Lipofectamine™(μl) according to manufacturer specifications and were used as a positive control. Due to toxicity observed with longer incubations, cells were only incubated for four hours with Lipofectamine™/pDNA complexes (2.5 μg pDNA/well), in serum free medium and then replenished with complete media.

Cells exposed to transfection agents were trypsinized (trypsin 0.25%-EDTA) and once detached, complete medium was added to inhibit trypsin. Cell suspensions were then transferred to 5 mL flow cytometry tubes and GFP expression in the transfected cells determined using a MoFlo cytometer (MoFlo BTS, DakoCytomation, Carpinteria Calif., USA) equipped with a 488 nm argon laser for excitation (model ENTCII-621, Coherent, Santa Clara Calif., USA). For each sample, 5,000-10,000 events were collected and fluorescence was detected through 510/20 nm (FL1) and 580/30 nm (FL2) band pass filters with photomultiplier tube voltages of 475 and 500, respectively. In addition, forward scatter (FSC) and side scatter (SSC) was used to establish a collection gate to exclude dead cells and debris. Signals were amplified in logarithmic mode for fluorescence and Summit software (v. 3.1, DakoCytomation) was used to determine the GFP positive events by a standard gating technique. The control sample was displayed on a dot plot (FL1 vs. FL2) and the gate drawn such that control cells were excluded. The percentage of positive events was calculated as the events within the gate divided by the total number of events, excluding dead cells and debris.

In the culture wells used to assess luciferase activity, culture medium was replaced with 100 μL of Glo Lysis Buffer (Promega, Madison Wis., USA) until complete lysis. Aliquots of 50 μL were transferred to 96-well white luminometer plates where an equal amount of Bright-Glo™ substrate (Promega) was added just prior to measurement on a Fusion luminometer (PerkinElmer, Wellesley Mass., USA). An aliquot of 25 μL was treated with Compat-Able™ Preparation Reagent Set (Pierce Biotechnology, Rockford Ill., USA) to remove interfering substances from the Glo Lysis Buffer prior to determining the protein content using BCA™ Protein Assay kit (Pierce Biotechnology). The relative light units (RLU) were normalized to the protein content of each sample.

Size of chitosan/pDNA complexes was determined by dynamic light scattering at an angle of 173° at 25° C., using a Malvern Zetasizer Nano ZS (Malvern, Worcestershire, UK). Samples were measured in triplicates using the refractive index and viscosity of pure water in calculations. The zeta potential (surface charge) was measured in duplicates with laser Doppler velocimetry at 25° C. on the same instrument and with the viscosity and dielectric constant of pure water for calculations. For both of the above measurements, nanoparticles were diluted 1:25 in PBS containing calcium and magnesium at pH of 6.5 and 7.1 and complexes were allowed to stabilize 30 minutes in the pH-adjusted PBS before reading.

### Depolymerization of Chitosan

By varying the amount of nitrous acid added to the different chitosans in solution (DDA ranging from 98 to 72%), chitosans with Mₙ close to the targets of 10, 40, 80 and 150 kDa (Table 2) were obtained. According to the literature, depolymerization using nitrous acid does not change the degree of deacetylation since nitrous acid attacks the amine groups, but not the N-acetyl moieties, and subsequently cleaves the β-glycosidic linkages, with no side reactions.

TABLE 2
Physicochemical Characteristics of Depolymerized Chitosans

| | Number-average | |
|---|---|---|
| Chitosans | molecular weight | Polydispersity, |
| DDA (%)^{a} | Mₙ (kDa)^{b} | PDI (M_{w}/Mₙ) |
| BST-98 | 120^{c} | 1.5 |
| 98% | 79 | 1.6 |
| | 39 | 1.6 |
| | 11 | 1.6 |
| BST-92 | 151 | 1.4 |
| 92% | 80 | 1.5 |
| | 38 | 1.6 |
| | 8 | 1.8 |
| BST-80 | 153 | 1.6 |
| 80% | 93 | 2.0 |
| | 38 | 2.6 |
| | 11 | 3.6 |
| BST-72 | 185 | 2.3 |
| 72% | 86 | 3.5 |
| | 39 | 4.0 |
| | 12 | 7.0 |

^{a}Determined by ¹H NMR.
^{b}Determined by gel permeation chromatography.
^{c}Not depolymerized, since this bulk chitosan has Mₙ of 120 kDa.

### Determination of Transfection Parameters

The mixing technique of chitosan and pDNA used to prepare nanoparticles, and the incubation conditions for transfection were first optimized using EGFP/flow-cytometric analysis of transfected cells. The best mixing technique was found to be adding chitosan over pDNA, pipetting up and down a few times and tapping the tube gently, compared to mixing under more vigorous vortex agitation. As for pre-transfection incubation conditions, there was no observed difference with incubation times in the range of 30-120 minutes, and incubation without agitation was found to give better transfection than incubation under vigorous agitation.

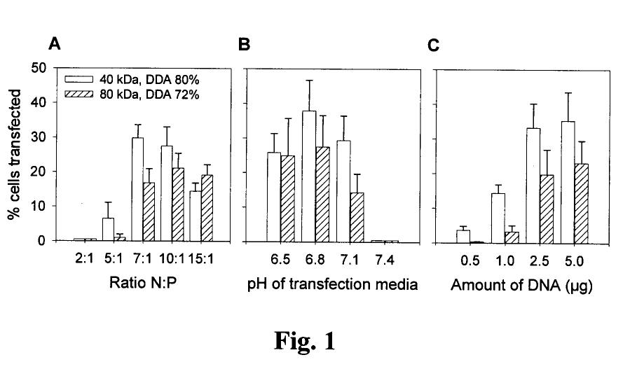

Prior to the analysis of the entire set of nanoparticles formulations using depolymerized chitosans, transfection parameters were optimized using only two chitosans selected in a pre-screening analysis (BST-80, 40 kDa and BST-72, 80 kDa). The N:P ratio of 2:1 with a transfection medium pH of 7.1 and 2.5 μg pDNA/well produced no transfected cells detectable by flow cytometry, while maximum transfection was found at 7:1 and 10:1 N:P ratios with a subsequent decrease at 15:1 (FIG. 1A). These results are consistent with those in the literature where excess chitosan is mixed with pDNA and the optimal N:P ratio can vary with DDA and MW (Kiang, 2004, supra).

Different pH of transfection media were tested, in the range of 6.5-7.4, remaining close to physiological values, even though some studies have reported transfection below pH 6.5 (Koping-Hoggard, 2004, supra; Romoren, 2003, supra). Comparable numbers of transfected cells were found for pH of 6.5 and 7.1, while a pH of 7.4 drastically lowered transfection for these chitosans used in the optimization experiments. (FIG. 1B).

A dose-dependant increase in number of cells transfected was seen with the amount of pDNA/well, up to 2.5 μg/well (FIG. 1C), where the transfection efficiency reached a plateau. Based on these results, N:P ratios of 5:1 and 10:1 and pH values of the transfection media of 6.5 and 7.1 as well as a dose of 2.5 μg pDNA/well were selected for further analysis of transfection using the library of depolymerized chitosans.

### Complex Size

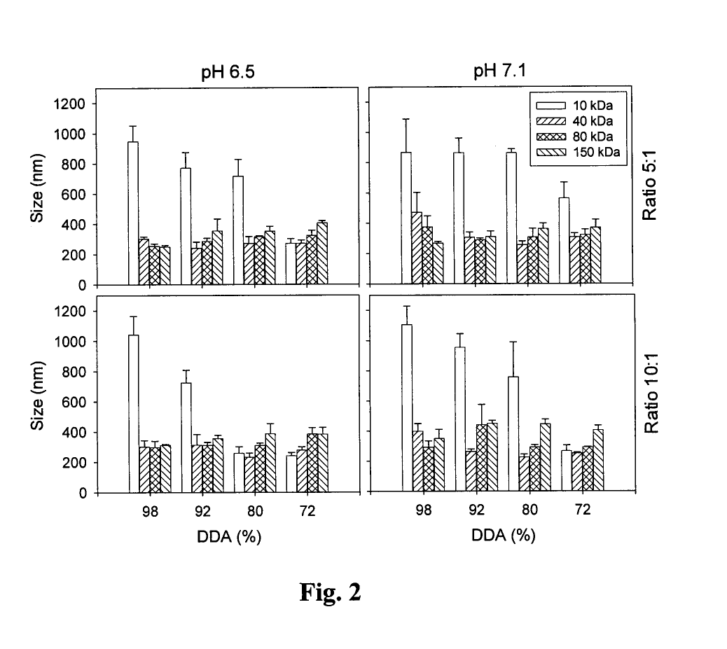

For most of the formulations tested, the size of the resulting complexes was found to be in the range 200-400 nm. However, 10 kDa chitosans led to the formation of larger complexes in the range of 600-1000 nm (FIG. 2). It has been reported that the reduction of length and charge of chitosan decreases its binding affinity to DNA and at sufficiently low MW, chitosan cannot fully condense DNA (Danielsen, S. et al., Biomacromolecules 5: 928-936, 2004). This is consistent with the increased size (1000 nm) for the complexes formed with chitosan of 10 kDa (FIG. 2). The results indicate that DDA, MW, N:P ratio and pH do not significantly influence complex size as long as the chitosan is large enough (MW>10 kDa) to fully condense DNA. Danielsen et al. (Danielsen, 2004, supra) obtained similar results suggesting that the size of the DNA condensates is mostly determined by the properties of the particular DNA molecule. Polydispersity of the chitosan used to complex DNA could also have an effect on the size of the resulting particles. For example, the large polydispersity obtained for low DDA/low MW depolymerized chitosans (Table 2) suggests the presence of larger chains in these chitosan solutions that could more effectively condense DNA and be responsible for some of the smaller particles seen with these low DDA/low MW chitosans (FIG. 2).

### Complex Zeta Potential

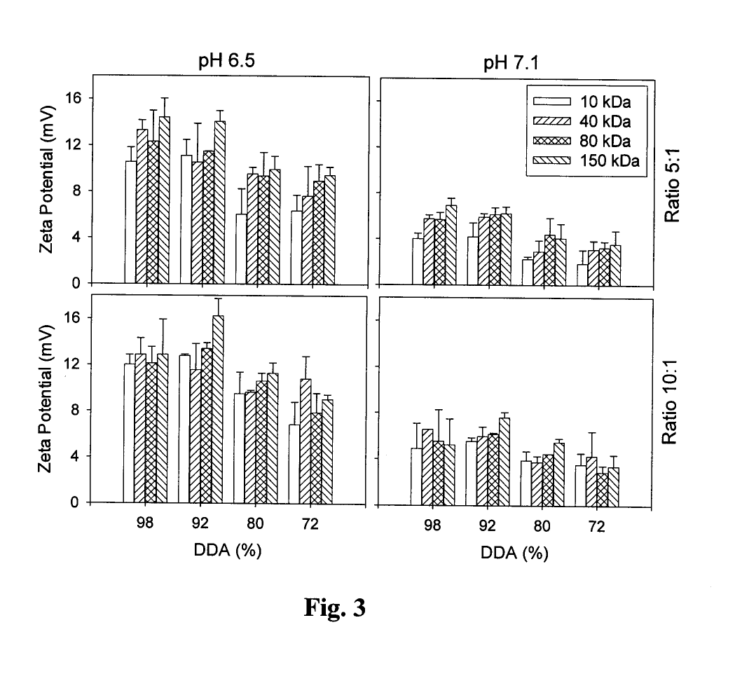

The zeta potential of the complexes was found to diminish with an increase of pH and to a lesser extent, with a decrease of chitosan's DDA, as expected (FIG. 3). Molecular weight did not significantly affect the zeta potential and no noticeable differences were observed comparing N:P ratios 5:1 and 10:1 (FIG. 3). As reported in several previous studies (Ishii, 2001, supra; Mao, 2001, supra), the zeta potential decreases when the pH rises, due to neutralization of amine groups on chitosan. The pKa of chitosan is reported to be 6.5 and the ionization state of the polymer is thus particularly sensitive to pH changes in the vicinity of pH 6.5, explaining the significant reduction in zeta potential observed when pH rises from 6.5 to 7.1. There was also a slight increase of the zeta potential as DDA increased (FIG. 3) due to the higher charge density of more deacetylated chitosans. Huang et al. (Huang, 2004, supra) found similar influences of DDA and MW on the zeta potential of chitosan particles. At high N:P ratio such as 5:1 and 10:1, the zeta potential appeared to reach a maximum as observed in previous studies (Kiang, 2004, supra; Mao, 2001, supra).

### In Vitro Transfection

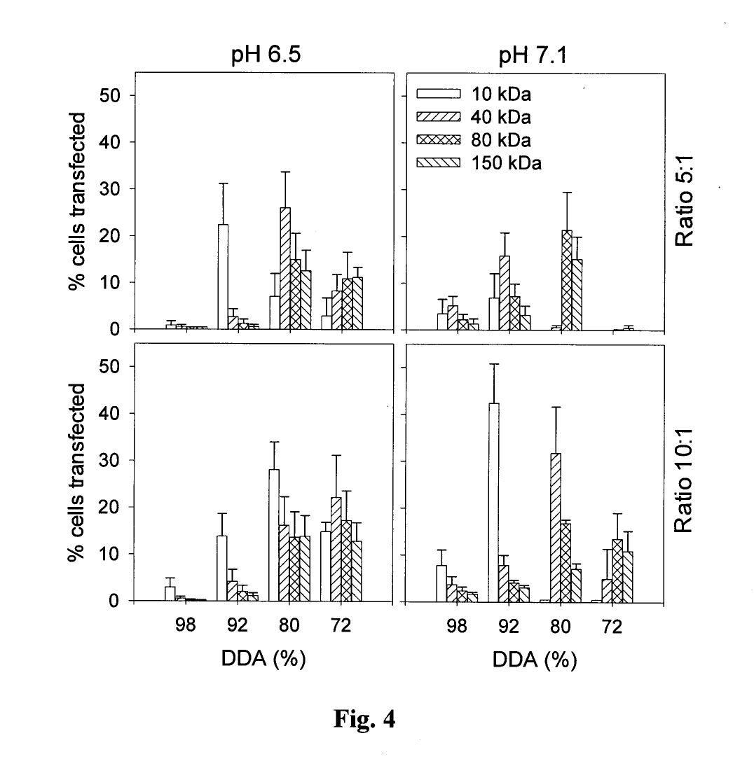

The percentage of transfected cells determined by flow cytometry was found to depend significantly on the type of complexes used, where some formulations resulted in as high as 40% of cells being transfected, whereas others revealed no transfection at all (FIG. 4). These results clearly demonstrate that transfection efficiency is highly sensitive to the MW, DDA, N:P ratio and pH of the transfection media.

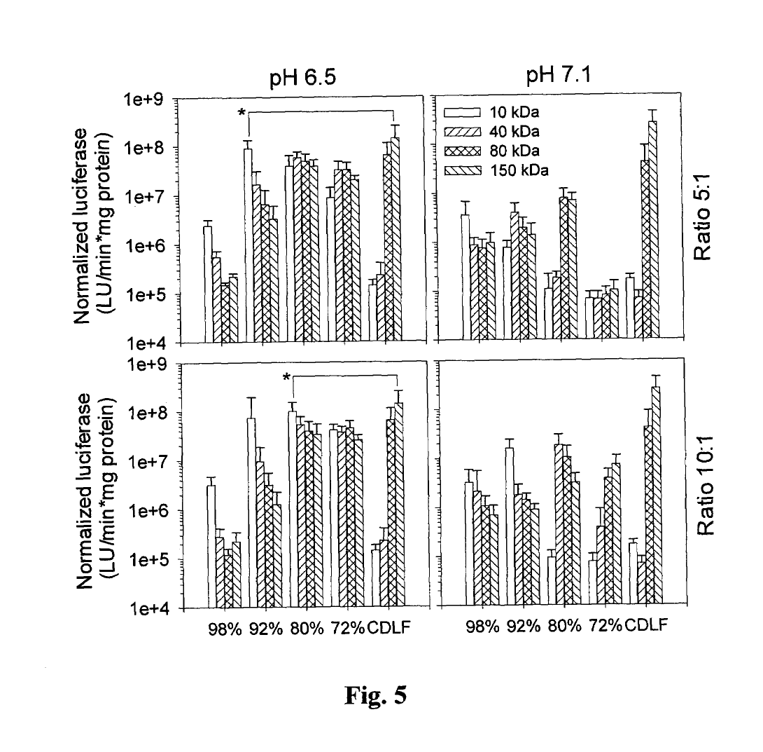

The level of luciferase expression was also found to vary strongly with the formulation parameters of the complexes. Many formulations with chitosans of MW between 8-185 kDa and DDA between 72%-92% resulted in levels of transgene expression approaching those of the positive controls (Lipofectamine™ and FuGENE 6). Most interestingly, two formulations at pH 6.5, namely 92-10-5 and 80-10-10 [DDA-MW-N:P ratio], were equivalent to our best positive control, FuGENE 6 (FIG. 5), since no statistically significant difference could be detected. FuGENE 6 is known to be a highly efficient commercial vector for in vitro transfection, clearly indicating that complexes produced with these two chitosan-based formulations achieved particularly high levels of transgene expression.

A more acidic transfection medium, and hence a higher zeta potential, was correlated with an increase of transgene expression for most of the formulations (FIG. 5). This direct correlation between lower pH and higher levels of gene expression has been previously observed (Koping-Hoggard, 2004, supra; Sato, 2001, supra). In media of lower pH, the zeta potential of the complexes increases due to chitosan ionization, effectively increasing complex stability extracellularly and enhancing their binding to negatively charged cell membranes and subsequent uptake (Huang, 2004, supra). Only 3 formulations did not behave in this manner, namely the complexes formed with chitosans of DDA=98% and with MW of 40, 80 and 120 kDa that had higher luciferase expression level at pH 7.1. However, microscopic observations revealed that cell morphology was altered after incubation with complexes using a 98% DDA chitosan at pH 6.5. Cytotoxicity of chitosan has been reported to increase with chitosan valence, or charge density (Richardson, 1999, supra), as would occur with higher DDA and lower pH. Some cytotoxicity may then appear for higher DDA and MW, particularly at acidic pH when these chitosans become more protonated. These findings are compatible with Huang et al. (2004, supra) who found attenuated cytotoxicity of chitosan when DDA was decreased, and less attenuation when MW was reduced suggesting that DDA has a greater effect than MW on cytotoxicity via its controlling effect on particle surface charge. An additional factor that may influence cytotoxicity is biodegradability since high DDA chitosans are more difficult to degrade due to the requirement of sequential N-acetyl-glucosamine units for binding to chitosan-degrading enzymes. However, in our transfection experiments, the same complexes (DDA=98% of MW 40, 80 and 120 kDa) used at higher pH 7.1 resulted in much better cell morphology and a higher level of expression, supporting a charge density-dependence influence on cytotoxicity rather than biodegradability. At pH 6.5, all formulations using 98% DDA chitosan were significantly less efficient than lower DDA formulations that reached higher levels of expression similar to the positive controls in several cases.

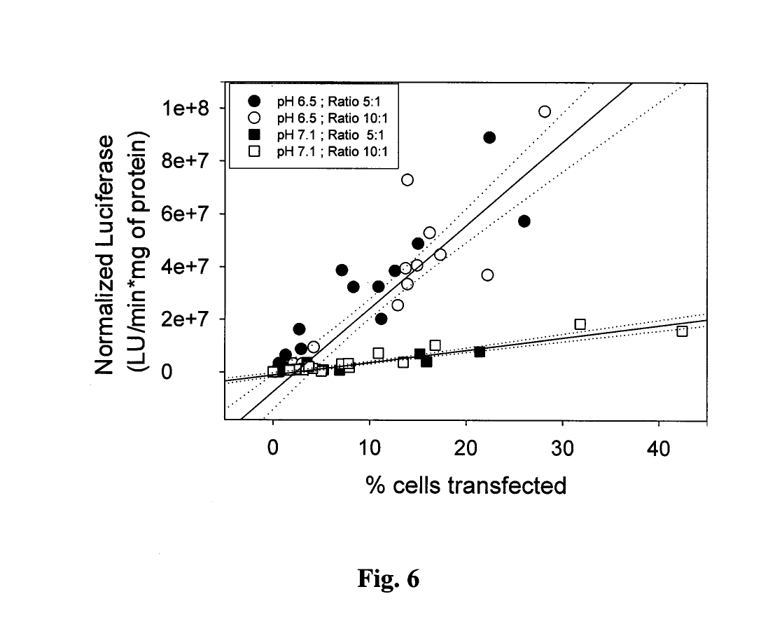

As can be seen by comparing the percentage of cells transfected (FIG. 4) and their level of transgene expression (FIG. 5), a similar percentage of cells transfected observed at pH 6.5 versus 7.1 does not correspond to the same level of transgene expression. Luciferase expression was much greater at pH 6.5 versus 7.1 even though the percentage of transfected cells could be similar. By grouping data according to pH, it was found to exist a linear relationship between the luciferase expression and the percentage of cells transfected, for both pH values but with a much higher slope at pH 6.5 (FIG. 6).

Interestingly, some of the larger complexes were able to transfect 293 cells quite efficiently and some of the smaller complexes were much less efficient (e.g. 92-10-5 (size of 770 nm) vs. 92-150-5 (350 nm) at pH 6.5 (i.e. with similar surface charge)). These results suggest that small complexes are not necessarily required for efficient transfection. Results from Koping-Hoggard et al. (Koping-Hoggard, 2001, supra) support this notion since transfection efficiency was not affected by different sizes of complexes (200-600 nm) obtained with the same formulation parameters (in their study, by decreasing chitosan and plasmid concentration at constant N:P ratio). Additional literature also suggests that the size of nanoparticles does not appear to be a dominant factor in cellular uptake.

### Influence of Formulation Parameters on Transfection Efficiency

The different levels of expression obtained with the various formulations can be rationalized in terms of the stability of the complexes. Complexes that are not sufficiently stable will dissociate when incubated in complete medium and will show little or no transfection. On the other hand, complexes that are too stable will not release DNA once inside the cells and will also show little or no transfection as well. Evidently, an intermediate stability that ensures that complexes do not disassemble in the transfection medium but will dissociate once internalized is required. The transfection parameters, DDA, MW, N:P ratio and pH can all affect complex properties and stability and are discussed below.

### Influence of DDA.

The DNA binding capacity of chitosan increases when its degree of deacetylation increases to create a higher charge density along the chain (Danielsen, 2004, supra; Kiang, 2004, supra). Thus chitosans with a DDA too low are unable to bind efficiently DNA and cannot form physically stable complexes to transfect cells (Koping-Hoggard, 2001, supra). As mentioned above, DDA also exerts a dominant influence on biodegradability where high DDAs are difficult to degrade. In this light, a recent study by Koping-Hoggard et al. (Koping-Hoggard, 2001, supra) suggested that endosomal escape of high MW chitosan-based complexes depended on enzymatic-degradation of chitosan (rather than a proton buffering capacity) that would occur less readily with high DDA chitosans. The resulting degradation fragments (oligo- and mono-saccharides) are hypothesized to increase endosome osmolarity and lead to membrane rupture. Thus, for highly deacetylated chitosan, reduced degradability could result in reduced endosomal escape.

### Influence of MW.

Binding affinity between oppositely charged macromolecules is strongly dependant on the valence of each molecule, with a low valence yielding only weak binding (Danielsen, 2004, supra). The reduction in chitosan valence for lower MW with shorter chains has been shown to reduce its affinity to DNA (Koping-Hoggard, 2003, supra). Although complex stability is desirable extracellularly, MacLaughlin et al. (MacLaughlin, 1998, supra) suggested that high MW chitosan can form complexes that are overly stable to transfect cells since they cannot be disassembled once inside the cell. Along these lines, Koping-Hoggard et al. (Koping-Hoggard, 2001, supra) found these more stable complexes could permit maximum gene expression after a relatively long period of 72 h. Thus formulations that are called “too stable” (high MW yet low DDA to be degradable) may release the plasmid several days post-transfection, resulting in delayed expression.

### Influence of N:P Ratio

Increasing the N:P ratio enhances chitosan binding to DNA. For the same DDA, a lower MW chitosan requires a higher N:P ratio to completely bind DNA. Similarly at equal MW, a lower DDA requires a higher N:P ratio to completely bind DNA (Kiang, 2004, supra; Koping-Hoggard, 2001, supra).

### Influence of pH.

Reduction of pH increases chitosan protonation as well as its binding affinity to DNA. An acidified medium also reduces possible aggregation of the complexes. Thus, complexes are in general more stable and more efficient to transfect cells in slightly acidic medium as shown by the results presented herein (FIG. 5).

### Coupling of Formulation Parameters to Determine Transfection Efficiency.

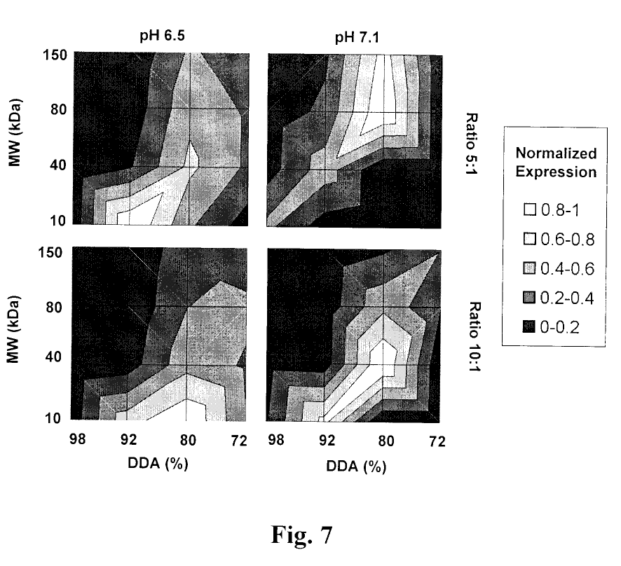

The combined effect of the formulation parameters (DDA, MW, pH, N:P ratio) is synthesized in FIG. 7. The numerous experiments with the chitosan library allowed us to produce contour plots of normalized transgene expression are presented as a function of DDA and MW for each pair of (pH, N:P ratio) tested. In each graph, transgene expression was normalized to the highest level of expression achieved at the corresponding (pH, N:P ratio) pair in order to specifically highlight the influence of DDA and MW. Interestingly, it appears that maximum transgene expression occurs for DDA:MW values that run along a diagonal from high DDA/low MW to low DDA/high MW. The exact location of this diagonal changes for different (pH, N:P ratio) pairs. Thus if one decreases/increases DDA, one must correspondingly increase/decrease MW to maintain maximal transgene expression. It was also observed that for a given DDA, a change in pH from 6.5 to 7.1 displaces the MW for the most efficient formulations towards higher MW because of the destabilizing effect of a pH increase that neutralizes chitosan. On the other hand, for a given DDA, a change in N:P ratio from 5:1 to 10:1 displaces the MW for the most efficient formulations towards lower MW, again probably because of the stabilizing effect of increasing chitosan concentration (N:P ratio).

The transition from an optimal formulation to a “too stable” formulation is conveniently illustrated by the behavior of 92% DDA chitosan at an N:P ratio of 5 and pH 6.5 (FIG. 5). As discussed above, this chitosan will not be efficiently degraded by endosomal enzymes because of its high DDA. These complexes based on 92% DDA chitosan show an important decrease in transgene expression, by a factor of 30, when MW increases from 10 to 150 kDa. These results clearly suggest that the complexes containing high DDA (92%) high MW (150 kDa) chitosan are too stable to transfect cells.

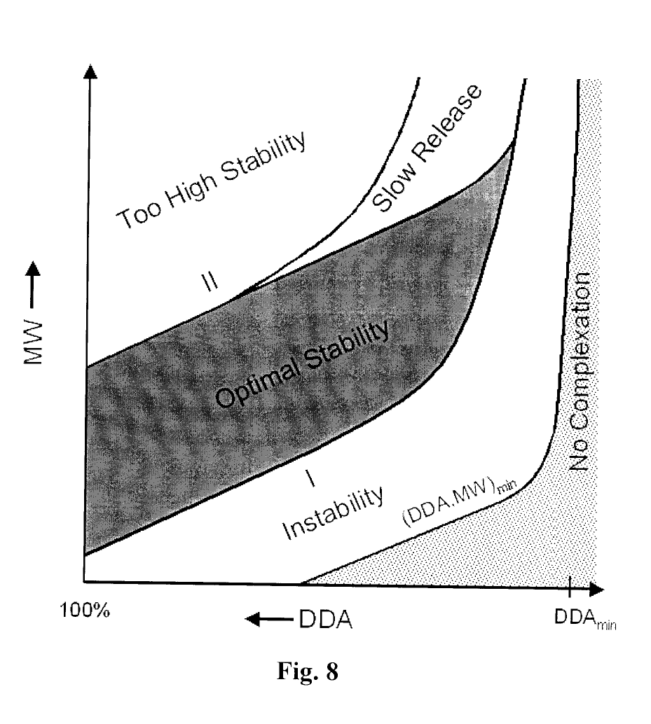

A schematic of complex stability as a function of DDA and MW synthesizes our findings into model that is predictive of transgene expression (FIG. 8). An optimal stability window exists that will yield efficient transfection for a DDA versus MW region dependent on particular values of pH and N:P ratio (“Optimal Stability” region in FIG. 8). A particular minimal DDA and MW combination (DDA,MW)ₘᵢₙ exists below which complexes will not form and, in addition, below a minimum DDA (DDAₘᵢₙ), MW cannot increase enough to complex DNA (“No Complexation” region in FIG. 8). On the opposite end of the spectrum, if the DDA and MW are too high, such complexes, once internalized, will not dissociate also resulting in no transfection (“Too High Stability” region in FIG. 8). For high molecular weight chitosans with lower DDA, the polymer is nonetheless degradable (degradation requiring a sequence of acetyl group) and DNA could be released slowly because of the high MW of the polymer, resulting in delayed expression (“Slow Release” region). On the other hand, if the MW and/or DDA are too low, the complexes are not sufficiently stable and they could dissociate too early in the transfection medium prior to binding and uptake (“Instability” region). The experimental results (FIG. 7) correspond closely to the characteristics of these schematized regions. Notably, an increase of N:P ratio stabilizes the complexes and will move the boundaries I and II of the “Optimal Stability” region downward. On the other hand, a decrease in pH of the transfection media will increase the cellular uptake through a higher surface charge, thus expanding the “Optimal Stability” region by only moving boundary I downward. In this case, the boundary II will not move since the intracellular degradability of the chitosan will not be altered for complexes once internalized.

### EXAMPLE 1

### Delivery and Expression of LacZ Transgene Using Chitosan/DNA Nanoparticles in Caco-2, HeLa and HT29 Cells

This example demonstrate that the chitosan/DNA gene delivery system of the present invention is efficient in multiple cell lines, showing its generality.

The efficacy of the system of the present invention was tested in three additional cell lines: Caco-2 (human colonic adenocarcinoma cells), HeLa (human epithelial cervical carcinoma cells) and HT29 (human colonic adenocarcinoma cells). Three chitosan formulations (chitosan 92-10-5, 80-10-10 and 80-80-5 [DDA (%)-Molecular Weight (kDa)-N/P ratio]) were used with a plasmid, pVax-LacZ which encodes for the enzymatic report protein β-galactosidase. Cells were incubated in 6 well-plates (37° C., 5% CO₂) in the presence of the chitosan/DNA nanoparticles for 18-48 hours prior to testing for transgene expression. Transgene expression was evaluated by a standard β-galactosidase assay. In summary, the culture cells were rinsed once with phosphate buffered saline (PBS), fixed (20% formaldehyde, 2% glutaraldehyde in PBS) for 10 minutes at room temperature, rinsed with PBS before staining with an X-Gal solution (400 mM potassium ferricyanide, 400 mM potassium ferrocyanide, 200 mM magnesium chloride and 20 mg/ml of a X-gal in N-N-dimethylformamide). Positive cells were counted manually 24 hours post-X-gal staining, using five random microscopic fields, in four separate experiments.

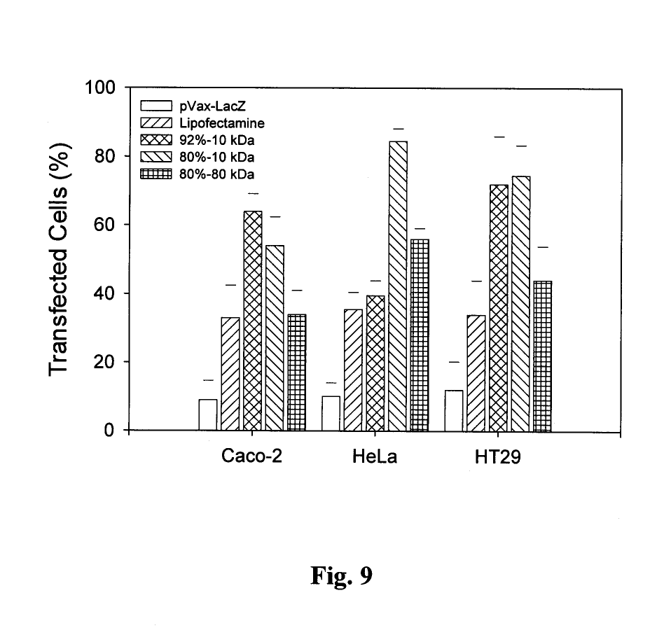

FIG. 9 shows that chitosan/DNA nanoparticle were efficient in delivering nucleic acids in the three cell lines tested in addition to HEK 293 presented herein above. In all cases, the chitosan/DNA nanoparticles were more efficient than DNA alone, and as efficient or even more efficient than the commercially available positive control lipofectamine, which however is toxic to cells. The chitosan formulations 92-10-5 and 80-10-10 were the most efficient in Caco-2 and HT29 cells, while 80-10-10 and 80-80-5 were best for HeLa cells. Overall, the chitosan formulation 80-10-10 achieved the best results.

### EXAMPLE 2

### Protein Expression and Antibody Production after Sub-Cutaneous Vaccination with Chitosan/DNA Nanoparticles

This example shows that the chitosan/DNA gene delivery system is efficient in vivo for therapeutic protein expression and for antibody production.

The gene delivery system using chitosan/DNA nanoparticles in accordance with the present invention was tested in vivo for FGF-2 protein expression and antibody production. Balb/c mice were injected sub-cutaneously on day 0, 7, 14, 21, and 49 using the same three chitosan formulations as in example 1 (92-10-5, 80-10-10 and 80-80-5 [DDA (%)-Molecular weight (kDa)-N/P ratio]) and the plasmid pVax-4sFGF-2 which encodes for the FGF-2 protein. Blood was drawn for each time point, as well as at sacrifice (day 63) and serum was collected for analysis. FGF-2 protein and antibody were detected in the serum using ELISA assays.

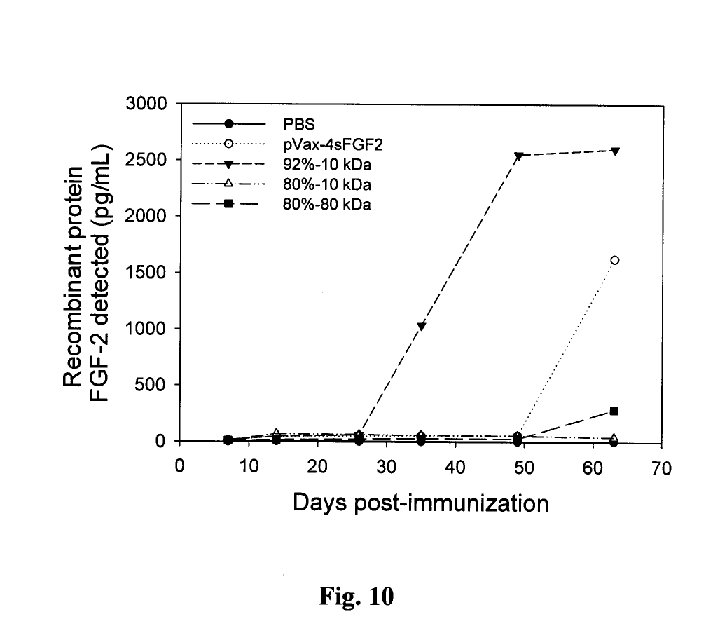

FIG. 10 shows a high level of protein expression for the chitosan 92%-10 kDa detected at 63 days, 1.6 times more than DNA alone (pVax-4sFGF2).

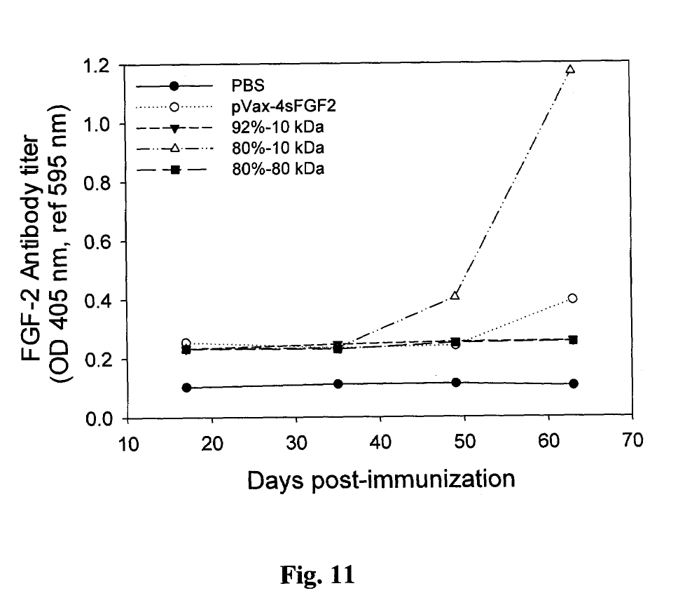

FIG. 11 shows a high level of antibody for the chitosan 80%-10 kDa measured at 63 days, 3 times more than DNA alone (pVax-4sFGF2)

The chitosan 80%-10 kDa is faster to induce an immunization in balb/c mice demonstrated by a high antibody titer detected at day 63 while the formulation 92%-10 kDa is much slower with a high level of proteins at day 63.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition comprising chitosan and a nucleic acid sequence for delivery of said nucleic acid sequence into cells, wherein the chitosan has a number-average molecular weight (Mₙ) between 8 kDa and 185 kDa and a degree of deacetylation between 72% and 92%.

2. The composition of claim 1 wherein said chitosan has a block distribution of acetyl groups.

3. The composition of claim 1, wherein said chitosan has a polydispersity between 1.4 and 7.0.

4. The composition of claim 1, wherein said composition has an amine:phosphate ratio of at least 5:1.

5. The composition of claim 4, wherein said amine:phosphate ratio is at least 7:1.

6. The composition of claim 8, wherein said amine:phosphate ratio is 10:1.

7. The composition of claim 1, further comprising a transfection media having a pH varying from 6.5 to 7.1.

8. The composition of claim 7, wherein the pH of said transfection media is 6.5.

9. The composition of claim 1, wherein said chitosan has been prepared by chemical or enzymatic hydrolysis.

10. The composition of claim 1, wherein the nucleic acid sequence is a deoxyribonucleic acid sequence.

11. The composition of claim 1, wherein the nucleic acid sequence is a ribonucleic acid sequence.

12. The composition of claim 1, wherein the nucleic acid sequence is a circular plasmid deoxyribonucleic acid sequence.

13. The composition of claim 1, wherein the composition is a dried powder.

14. The composition of claim 1 wherein the composition is a particular suspension in aqueous media.

15. A method for delivering a nucleic acid sequence into a cell comprising the step of contacting the composition of claim 1 with said cell.

16. The method of claim 15, wherein said cell is an isolated cell in culture.

17. The method of claim 16, wherein said isolated cell is a primary cell, a transformed cell or an immortalized cell.

18. The method of claim 15, wherein said nucleic acid sequence codes for a polypeptide.

19. The method of claim 15, wherein said nucleic acid sequence is an antisense nucleic acid sequence.

20. The method of claim 15, wherein said nucleic acid sequence is a small interfering ribonucleic acid sequence.

21. The method of claim 15, wherein said cell is a mammalian cell.

22. The method of claim 21, wherein said mammalian cell is a non-human mammalian cell.

23-42. (canceled)
